(19) United States
(12) Patent Application Publication
(10) Pub. No.: US 2007/0049941 A1
(43) Pub. Date: Mar. 1, 2007

(54) PLATE WITH STABILIZATION

(75) Inventor: Jeffery Thramann, Longmont, CO (US)

Correspondence Address: HOLLAND & HART, LLP, P.O BOX 8749, DENVER, CO, 80201, US

(73) Assignee: Lanx, LLC, Boulder, CO (US)

(21) Appl. No.: 11/420,417

(22) Filed: May 25, 2006

Related U.S. Application Data
(60) Provisional application No. 60/711,352, filed on Aug. 25, 2005.

(51) Int. Cl. A61F 2/30

(52) U.S. Cl. 606/69

(57) ABSTRACT

A spinal fusion plate includes a means to support an adjacent vertebral segment to inhibit the adjacent vertebral segment from further degeneration. The means to support includes an attachment to an associated artificial disc or nucleus replacement, an extension, or an attachment to a bone anchor. In each case, the attachment is moveable in relation to the fusion plate to allow flexion and extension.

### RELATED APPLICATIONS AND CLAIM OF PRIORITY

The present invention claims priority to U.S. Provisional Patent Application Ser. No. 60/711,352, filed Aug. 25, 2005, titled Plate with Stabilization, the disclosure of which is incorporated herein by reference as if set out in full.

### FIELD OF THE INVENTION

The present invention relates to spinal corrective surgery and, more particularly to plate with a spinal stabilization device to inhibit disc degeneration and adjacent disc degeneration.

### BACKGROUND OF THE INVENTION

The vertebrae of the human spine are arranged in a column with one vertebra on top of the next. Between each vertebra exists an intervertebral disc that transmits force between adjacent vertebrae and provides a cushion between the adjacent vertebrae.

Sometimes, back pain is caused by degeneration or other deformity of the intervertebral disc (“diseased disc”). Conventionally, surgeons treat diseased discs by surgically removing the diseased disc and inserting an implant in the space vacated by the diseased disc, which implant may be bone or other biocompatible implants. The adjacent vertebrae are then immobilized relative to one another. Eventually, the adjacent vertebrae grow into one solid piece of bone.

For example, a conventional method to fuse vertebrae together includes a bone graft and a plate to stabilize the device. The current process of inserting a bone graft and fusing the adjacent vertebrae will be explained with referring to FIGS. 1 and 2. FIG. 1 shows two adjacent vertebrae 102 and 104. Located between vertebrae 102 and 104 is an intervertebral space 106 partially filled by an implant 108. When the implant 108 is first inserted into the intervertebral space 106, the adjacent vertebrae 102 and 104 are manually kept apart by the surgeon using, for example, a retracting device (not shown). As shown in FIG. 2, once the implant 108 is placed, the surgeon releases the adjacent vertebrae 102 and 104 allowing them to squeeze the implant 108 and hold the implant 108 in place.

To immobilize the vertebrae 102 and 104 with the implant 108 in place, the surgeon next applies a plate 202 over the adjacent vertebrae 102 and 104. Plate 202 may have a central viewing window 204 and one or more screw holes 206, in this example four screw holes 206a-206d are shown. Four bone screws (which will be identified by reference numerals 208a-208d) would be screwed into the vertebrae using the screw holes 206 to anchor the cervical plate to the vertebrae and immobilize the vertebrae with respect to one another.

Immobilizing the superior and inferior vertebrae with a bone graft in the intervertebral disc space prompts fusion of the superior and inferior vertebrae into one solid bone. As can be appreciated, fusing vertebral bodies like this limits the range of motion of the spine. Moreover, fusing vertebral bodies together may accelerate or contribute to the degeneration of adjacent discs. Sometimes adjacent discs may already have some disc degeneration, but the degeneration is not sufficient to justify additional fusing.

Against this background, it would be desirous to develop a fusion technology that provided some support for adjacent discs to inhibit any or additional degeneration of those discs.

### SUMMARY OF THE INVENTION

The present invention provides a dynamic spinal stabilization system. The dynamic spinal stabilization system comprises a spinal fusion plate to fuse a vertebral segment. An artificial disc is implanted into a intervertebral disc space proximate the vertebral segment being fused. A movable member couples the spinal fusion plate and the artificial disc to provide support in relate to flexion and extension of the spine.

The present invention also provides a dynamic spinal stabilization system comprising a spinal plate to fuse adjacent vertebral bodies. The plate has an extension that traverses the spinal column to provide support to a superior or inferior vertebral body. The extension is pivotally connected to the plate.

The present invention also provides a dynamic spinal stabilization system comprising a spinal plate to promote fusing adjacent vertebral bodies into a single bone mass. An anchor is coupled to a vertebral body proximate the fused adjacent vertebral bodies. A movable member couples the anchor to the plate.

The present invention also provides an intervertebral disc support clip. The support clip includes a first surface adjacent an inferior surface of a superior vertebral disc and a second surface adjacent a superior surface of an inferior vertebral disc. The first surface and second surface are connected by a wall traversing intervertebral disc space of an intervertebral disc to be supported. The support clip is formed of a biased material so first surface and second surface tend away from each other and distract the intervertebral disc space.

The foregoing and other features, utilities and advantages of the invention will be apparent from the following more particular description of a preferred embodiment of the invention as illustrated in the accompanying drawings.

### BRIEF DESCRIPTION OF THE DRAWINGS

The accompanying drawings, which are incorporated in and constitute a part of this specification, illustrate embodiments of the present invention, and together with the description, serve to explain the principles thereof. Like items in the drawings are referred to using the same numerical reference.

FIG. 1 is a block diagram of adjacent vertebrae with an implant in an intervertebral disc space;

FIG. 2 is an elevation view of adjacent vertebrae with a plate attached to adjacent vertebrae to facilitate fusion of the adjacent vertebrae;

FIG. 3 is a cross-sectional view of FIG. 2 showing an adjacent intervertebral disc space and vertebral body;

FIG. 4 is a cross-sectional view of another embodiment consistent with the present invention;

FIG. 5 is a cross-sectional view of another embodiment consistent with the present invention;

FIG. 6 is an elevation view of another embodiment of the present invention;

FIG. 7 is an elevation view of another embodiment of the present invention;

FIG. 8 is an elevation view of another embodiment of the present invention;

FIG. 9 is a cross-sectional view of an intervertebral disc support;

FIG. 10 is an anterior view of the device of FIG. 9; and

FIG. 11 is a top elevation view of the device.

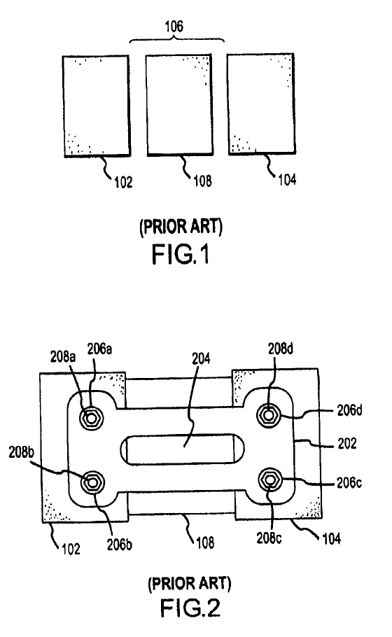

### DETAILED DESCRIPTION

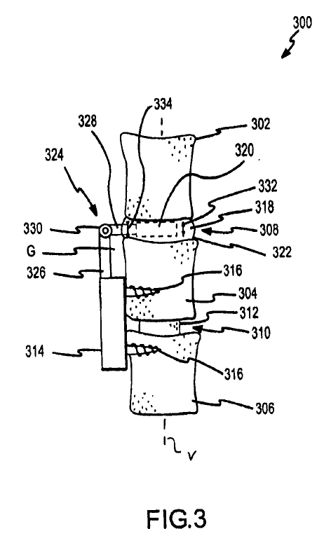

The present invention will now be described with reference to the figures. Referring first to FIG. 3, a cross-sectional view a spinal segment 300 is shown. Spinal segment includes a first vertebral body 302, a second vertebral body 304, and a third vertebral body 306. Residing between first vertebral body 302 and second vertebral body 304 is first intervertebral disc space 308. First vertebral body 302 may be superior to second vertebral body 304. Second vertebral body 304 may be superior to third vertebral body 306. Residing between second vertebral body 304 and third vertebral body 306 is second intervertebral disc space 310.

Further shown in FIG. 3 is a bone graft 312 residing in second intervertebral disc space 310. Bone graft 312 may be constructed out of a number of biocompatible materials, such as, for example, milled bone, PEEK material, titanium, resorbable material, or the like. A plate 314 traverses a vertical axis V of the spinal column and is coupled to second vertebral body 304 and third vertebral body 306 to stabilize the bodies about intervertebral disc space 310 to prompt bone growth in intervertebral disc space 310 around bone graft 312. Plate 314 is anchored to second vertebral body 304 and third vertebral body 306 by bone screws 316. Plate 314 may be constructed from any biocompatible material such as, for example, NiTi, other shaped memory alloys, titanium, resorbable material, PEEK material, or the like. Eventually, bone growth in disc space 310 fuses vertebral body 304 and vertebral body 306 into a solid piece of bone.

The fusion of second vertebral body 304 to third vertebral body 306 operates to alleviate pain, but frequently adjacent discs, such disc 318 associated with intervertebral disc space 308, are experiencing some degeneration. Fusing second vertebral body 304 and third vertebral body 306 may provide additional stress on disc 318 because of the increased load and motion intervertebral disc 318 must support due to the fusing that accelerates or initiates degeneration of disc 318, a.k.a adjacent disc disease. While possible to fuse first vertebral body 302 and second vertebral body 304, it is a less than desirable solution because of the decreased range of motion and the additional stress placed on intervertebral discs not shown in FIG. 3.

Still with reference to FIG. 3, an artificial member 320 may replace biological disc 318, completely or partially. For example, artificial member 320 may be an artificial disc or artificial member 320 may be a nucleus replacement. If artificial member 320 is an artificial disc, it may be constructed in accordance with conventional artificial discs, such as those described in U.S. Pat. No. 6,770,094, titled INTERVERTEBRAL DISC PROSTHESIS, issued Aug. 3, 2004, to Fehling et al., incorporated herein by reference as if set out in full, U.S. patent application Ser. No. 10/641,530, titled SHAPED MEMORY ARTIFICIAL DISC AND METHODS OF ENGRAFTING THE SAME, filed Aug. 14, 2004, incorporated herein by reference as if set out in full, and a U.S. Pat. No. 6,881,228, titled ARTIFICIAL DISC IMPLANT, issued Apr. 19, 2005, to Zdeblick et al., incorporated herein by reference as if set out in full. Alternatively, artificial member 320 may be a nuclear implant, NiTi implant, or support, or the like. Generically, artificial member 320, nuclear implants, NiTi implants, supports and the like are generically referred to as disc support devices.

Artificial member 320 would provide resistance to further degeneration, but fusion of second vertebral body 304 and third vertebral body 306 still provides additional stress. To reduce the stress, a member 324 (or several members 324) may be provided extending from plate 314 to artificial member 320. Member 324 should have some elasticity to allow flexion, extension, and preferably rotation to allow range of motion for the spinal segment 300. For example, member 324 may be provided, in part, as a shaped memory alloy (SMA), an elastic biocompatible polymer, a ball and socket joint, a hinge style connection, other modular connection, or the like.

Member 324 will have a plate extension 326 traversing a portion of spinal segment 300 in along the vertical axis. Plate extension 326 extends from plate 314 to first intervertebral disc space 308. Plate Extension 326 may be flush with spinal segment 300 to provide additional support or a gap G may be provided between spinal segment 300 and extension 326. Member 324 also includes a disc extension 328, which extends perpendicular or somewhat perpendicular depending on anatomical conditions from vertical axis V. Disc extension 328 extends from artificial member 320 through annulus 322 (unless annulus 322 has been removed) to a pivotal connection 330 connecting disc extension 328 and plate extension 326. Pivotal connection 330 allows for movement between disc extension 328 and plate extension 326. Pivotal connection 330 may be similar to a hinge, an elastic polymer, a ball and socket joint, a shaped memory alloy such as NiTi, a resin or the like to allow some rotational movement. Moreover, if member 324 is sufficiently flexible, pivotal member 330 may be removed.

As mentioned above, member 324 should have some elasticity to allow for flexion, extension, and rotation of spinal segment 300. The elasticity may be provided only in pivotal connection 330. Alternatively or in combination, plate extension 326 or disc extension 328 may be made of an elastic biocompatible material. Member 324 should be sufficiently elastic to allow movement, which inhibits bone growth and fusion of first vertebral body 302 and second vertebral body 304, by sufficiently rigid to provide support to artificial disc 320, which inhibits further degeneration. For example, if spinal segment 300 was associated with the cervical region of the spine, and the plate 314 was implanted on the anterior side of the spine, flexion of the neck area would cause distraction 332 of the posterior disc space in first intervertebral disc space 308 and compression 334 of the anterior disc space. In this case, member 324 would provide support and inhibit the stress on artificial disc 320. Further, extension of the neck would cause the opposite reaction and member 324 would provide support to artificial disc 320 by resisting over extension.

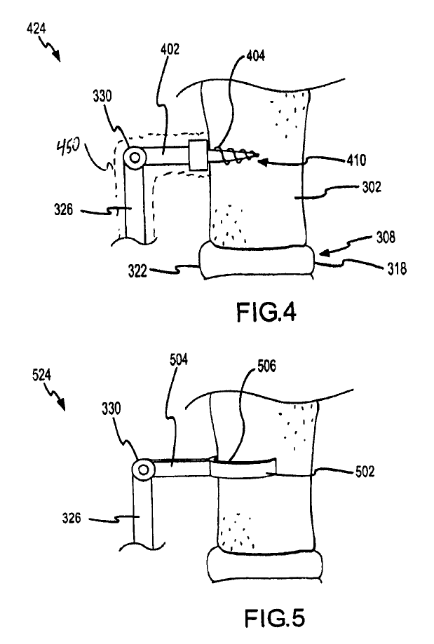

In some instances, biological disc 318 may not have sufficiently degenerated to justify using artificial member 320. But, it would still be desirable to provide support to biological disc 318 to inhibit degeneration of that disc. As shown in FIG. 4, member 424 is similar to member 324 and still has plate extension 326 and pivotal connection 330, but instead of disc extension 328, member 424 has an anchor extension 402 attached to an anchor 404, which as shown is a vertebral body screw. As shown in this case, anchor 404 as a vertebral body screw is threaded into first vertebral body 302. Member 424 could be used with artificial member 320 instead of member 324. In other words, instead of having disc extension 328 associated with member 324, member 424 with vertebral body screw extension 402 could be used even if an artificial member 320 is implanted in space 308 Anchoring member 424 to an adjacent vertebral body using a vertebral pedicle screw type anchor 404 has the potential that the screw may backout over time from the vertebral body. Therefore, if an anchor 404, such as a screw, is used, the anchor should be provided with a mechanism 410 to promote bone growth, using, for example, conventional bone fusion cage technology, coating the screw with bone growth material, providing striations on the screw, a combination thereof, or other conventional methods and devices to promote bone growth. Moreover, member 424 may be inhibited from movement a sufficient length of time to promote anchor 404 fusing to vertebral body 302. For example, if member 424 is formed of shaped memory alloy, it could be implanted in the stiff or non-elastic so the device is inflexible, which provides time for anchor 404 to fuse. Alternatively, member 424 can be captured in a stiff resorbable cast 450. Stiff resorbable material inhibits movement allowing anchor 404 to fuse to vertebral body 302. Resorbable cast 450 breaks down over time allowing member 424 to become flexible after sufficient time for fusion of anchor 404.

Referring now to FIG. 5, a member 524 is provided. Member 524 comprises plate extension 326 and pivotal connection 330, but has a clamp 502 and clamp extension 504. Clamp 502 connects about first vertebral body 302 instead of using a screw or fixed connector/anchor to connect member 524 to first vertebral body 302. Clamp 502 may have an inner surface 506 adjacent vertebral body 302. Inner surface 506 should may be movable or slidable relative to vertebral body 302. In this instance, inner surface 506 may comprises a low friction biocompatible material. Allowing vertebral body 302 and clamp 502 to move relative to each other allows more freedom of motion between vertebral body 302 and 304 to inhibit bone growth. But the clamp can support both flexion and extension of the spine.

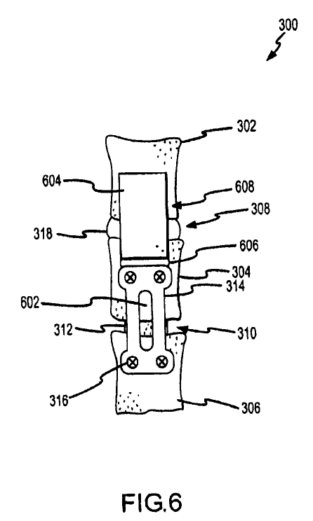

Referring now to FIG. 6, spinal segment 300 with a stabilization device 600 is provided consistent with the present invention. FIG. 6 shows spinal segment in an elevation view. For example, if spinal segment 300 was associated with the cervical region, FIG. 600 would be a view of the spine looking down through the neck. Spinal segment 300 includes, in order, first vertebral body 302, first intervertebral disc space 308, second vertebral body 304, second intervertebral disc space 310, and third vertebral body 306. Similar to the above, a fusion procedure has been performed such that bone graft 312 resides in second intervertebral disc space 310 promoting fusion between second vertebral body 304 and third vertebral body 306. Plate 314 is coupled to second vertebral body 304 and third vertebral body 306 using bone screws 316. Plate 314 may have a window 602 or the like through which space 310 and graft 312 may be viewable.

As mentioned above, fusing second vertebral body 304 and third vertebral body 306 causes disc 318 to support more stress, due to increased movement, rotation, or load across the disc space. To assist disc 318 support the increased stress, an extension plate 604 may be coupled to plate 314. Extension plate 604 is attached to plate 314 with a flexible/pivotal connection 606. Extension plate 604 may be a flat plate, shaped similar to plate 314, or shaped to cradle vertebral body 302. If shaped to cradle vertebral body 302, extension plate 604 may be provided with an inner surface 608 made of a low friction material to accommodate relative movement between vertebral body 302 and extension plate 604. Flexible/pivotal connection 606 may be, for example, a hinge, ball (or rod) and socket joint, shaped memory alloy such as NiTi, a gel, resin, or the like to support pivotal movement. Flexible/pivotal connection 606, however, may comprise an elastic polymer to support both pivotal movement between first vertebral body 302 and second vertebral body 304 as well as rotational movement. Given the example is an anterior cervical view of stabilization device 600, when a patient bends the neck towards the chest, extension plate 604 would contact first vertebral body 302 and provide some support so some of the additional stress is diverted from disc 318. Notice, while shown a separate parts for convenience, extension plate 604, flexible/pivotal connection 606, and plate 314 can be incorporated into a single unit. Stabilization device 600 could be used on the posterior side of the spinal segment 300, but would have to be designed to accommodate other spinal features, such as, for example, the spinous process.

Referring now to FIG. 7, a stabilization device 700 is shown. Stabilization device 700 is similar to device 600 and the similar parts will not be explained herein. Stabilization device 700 includes plate 314, one or more flexible/pivotal connection(s) 606 connecting one or more support legs 702 to plate 314. Support legs 702 operate similar to extension plate 604 explained above.

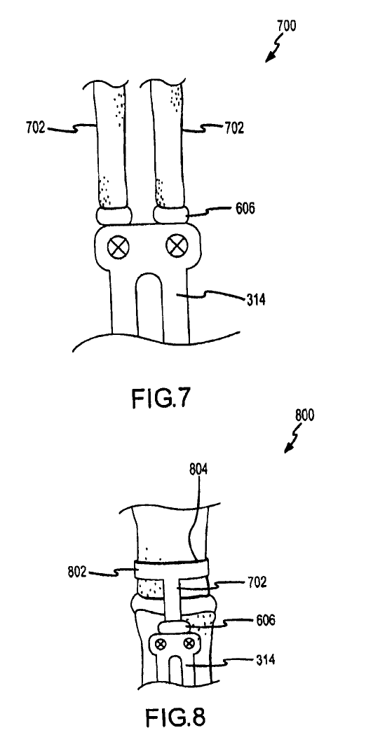

Referring now to FIG. 8, a stabilization device 800 is shown. Stabilization device 800 comprises a flexible/pivotal connection 606 coupling a support leg 702 to plate 314. At an end of leg 702 distal to connection 606, a horizontal support member 802 is provided. Horizontal is in relation to vertical axis V, but one of skill in the art would now recognize vertical and horizontal are relative to the position of a body. Horizontal support member 802 traverses vertebral body 302. Optionally, and as shown, horizontal support member 802 may wrap around a portion of vertebral body 302 to cradle vertebral body 302 to provide support in both flexion and extension. An inner surface of horizontal support member 802 may be made of a low friction material to facilitate relative movement.

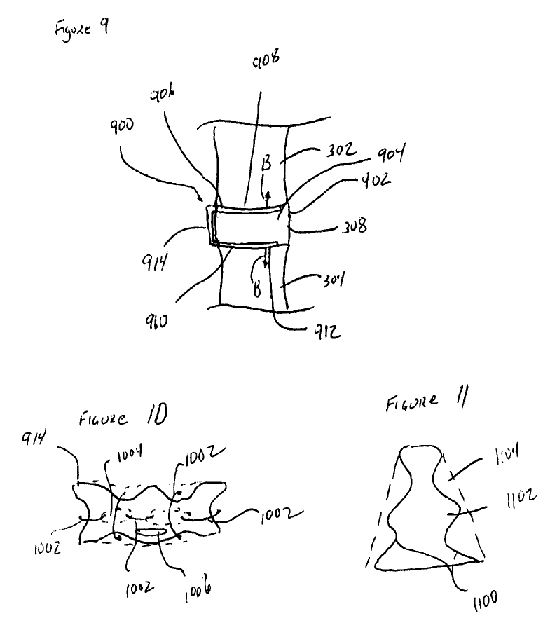

Referring to FIG. 9, A cross-sectional view of first vertebral body and second vertebral body across first intervertebral disc space 308 is provided with a disc support clamp 900. Disc space 308 is occupied by a biological disc comprising an annulus 902 and disc nucleus 904 (alternatively the biological disc could be replaced by an artificial disc 320). Disc support clamp 900 includes a first clip surface 906 proximate an inferior surface 908 of superior disc 302 and a second clip surface 910 proximate a superior surface 912 of inferior disc 304. Clip surfaces 906 and 910 are shown traversing a portion of the distance between annulus 902 and disc surface 908 and 912 respectively. Clip surfaces 906 and 910 could extend more or less across the vertebrae as desired. A wall 914 extends from clip surface 906 to 910 traversing disc space 308. Clip surface 906 and clip surface 910 are biased in a direction indicated by arrows B. Support clip 900 could be made out of a spring metal, a shaped memory alloy, polymer, or the like. Support clip 900 may be connected to a fusion plate 314 using a member 324 similar to device 300. Alternatively, support clip 900 may be a separate device. If separate, support clip would need to be anchored. Support clip 900 could be anchored using a natural ligament, if available. Alternatively, support clip 900 could be sutured using sutures 1002, see FIG. 10 below. In this case, support clip 900 would be designed with suture windows 1004 shown in phantom. Still alternatively, support clip could be anchored with a reconstructed ligament if the biological ligament is severed. To assist in reconstructing the ligament, wall 914 may be provided with an insert 1006. Insert 1006 may be constructed as a buckle, slot, or the like. To reconstruct the ligament, a scaring material would be attached to insert 106, such as a cotton material or mesh material. Insert 1006 may be also used to anchor the clip to a disc annulus by placing scarring material between the clip and the disc annulus.

Referring to FIG. 11, clip surface 906 is shown. Clip surface 906 could be substantially the same size as inferior surface 908, but preferably it is shaped to allow annulus 902 to remain in contact with inferior surface 908. Surface 906, of which surface 910 may be identical and is not shown, has outer edge 1100. Edge 1100 may have a curved shape 1102 (or multiple curves as actually shown) or a straight shape. Using curved or straight edges, surface 90 6 could have any geometric or random configuration. Referring to FIG. 10, an anterior view of support clip 900 is shown. As can be seen, wall 914 may be shaped similar to annulus 902. Alternatively, wall 914 may have suture windows 1004. Windows 1004 placements as shown are exemplary and non-limiting.

While the above figures show a plate extending over one level, one of ordinary skill in the art will recognize on reading the disclosure that the present invention would be useful for multiple level fusions. Moreover, although the stabilization device is depicted extending from a single end of the plate, one of ordinary skill in the art on reading the disclosure would understand that the present invention could have stabilization devices extending from multiple connection points, i.e, the superior and inferior direction.

While the invention has been particularly shown and described with reference to embodiments thereof, it will be understood by those skilled in the art that various other changes in the form and details may be made without departing from the spirit and scope of the invention.

1. A dynamic spinal stabilization system comprising:
a spinal fusion plate for fusing a vertebral segment;
at least one artificial disc for insertion into an intervertebral disc space proximate the vertebral segment; and
at least one member movably coupling the spinal fusion plate and the artificial disc to provide support in relation to at least one of flexion and extension of the spine, the member comprises a plate extension and a disc extension.

2. The dynamic spinal stabilization system according to claim 1, wherein the spinal fusion plate is an anterior cervical plate.

3. The dynamic spinal stabilization system according to claim 1, further comprising a bone graft to be inserted into an intervertebral space associated with the vertebral segment to be fused.

4. The dynamic spinal stabilization system according to claim 1, wherein the member further comprises a pivotal connection between the plate extension and the disc extension.

5. The dynamic spinal stabilization system according to claim 4, wherein the pivotal connection is selected from a group of pivotal connections consisting of: a hinge, a ball and socket, a gel, a resin, a polymer connection, or a shaped memory alloy.

6. The dynamic spinal stabilization system according to claim 1, wherein the spinal fusion plate is constructed from a resorbable material.

7. The dynamic spinal stabilization system according to claim 1, wherein the at least one artificial disc comprises at least two artificial disc for insertion into a superior intervertebral disc space and an inferior intervertebral disc space and the at least one member comprises at least two members wherein the a first member extends in a superior direction and a second member extends in an inferior direction.

8. A dynamic spinal stabilization system, comprising:
a spinal plate to couple a superior vertebral body and an inferior vertebral body;
at least one extension coupled to the spinal plate, the at least one extension to traverse a spinal column to a proximate vertebral body, the proximate vertebral body proximate at least one of the superior vertebral body or the inferior vertebral body; and
the at least one extension coupled to the spinal plate by a pivotal connection.

9. The dynamic spinal stabilization system of claim 8, wherein the at least one extension comprises a plate.

10. The dynamic spinal stabilization system of claim 8, wherein the at least one extension comprises at least one leg.

11. The dynamic spinal stabilization system of claim 8, wherein the at least one extension cradles the proximate vertebral body.

12. The dynamic spinal stabilization system of claim 8, wherein the at least one extension comprises a low friction material to facilitate relative motion between the at least one extension and the proximate vertebral body.

13. The dynamic spinal stabilization system of claim 8, wherein the at least one extension comprises a leg and a horizontal extension.

14. The dynamic spinal stabilization system of claim 13, wherein the horizontal extension slidably clamps the proximate vertebral body.

15. The dynamic spinal stabilization system of claim 8, wherein the pivotal connection pivots to support flexion and extension of the spine.

16. The dynamic spinal stabilization system of claim 15, wherein the pivotal connection pivots to support rotating and bending of the spine.

17. A dynamic spinal stabilization system, comprising:
a spinal plate to promote fusing adjacent vertebral bodies into a single bone mass;
an anchor coupled to a vertebral body proximate the adjacent vertebral bodies; and
a member movably coupling the spinal plate and the anchor, the member comprising at least a plate extension extending between the spinal plate and the anchor.

18. The dynamic spinal stabilization system of claim 17, wherein the anchor comprises a pedicle screw.

19. The dynamic spinal stabilization system of claim 18, wherein the pedicle screw further comprises a fusion cage to promote bone growth to inhibit backout.

20. The dynamic spinal stabilization system of claim 17, wherein the member comprises a joint coupling the spinal plate and the member.

21. The dynamic spinal stabilization system of claim 20, wherein the joint is one of a hinge, a ball and socket, a gel, a resin, a polymer, or a shaped memory alloy.

22. The dynamic spinal stabilization system of claim 20, wherein the joint supports flexion, extension, bending, and rotational movement of the spine.

23. The dynamic spinal stabilization of claim 17, wherein the member comprises a joint coupling the member and the anchor.

24. The dynamic spinal stabilization of claim 23, wherein the joint is one of a hinge, a ball and socket, a gel, a resin, a polymer, or a shaped memory alloy.

25. The dynamic spinal stabilization of claim 23, wherein the joint supports flexion, extension, bending, and rotational movement of the spine.

26. The dynamic spinal stabilization of claim 17, further comprising a cast about member to inhibit the member from moving for a period of time sufficient to allow the anchor to fuse to the to the proximate vertebral body.

27. The dynamic spinal stabilization of claim 26, wherein the cast comprises resorbable material.

28. The dynamic spinal stabilization of claim 17, wherein the member comprises a shaped memory alloy.

29. The dynamic spinal stabilization of claim 29, wherein the shaped memory is transitioned to an elastic phase after the anchor fuses to the proximate vertebral body.

30. A support for an intervertebral disc, comprising:
a first surface to reside between an inferior surface of a superior vertebral body and a disc annulus;
a second surface to reside between a superior surface of an inferior vertebral body and the disc annulus; and
a wall connecting the first surface and the second surface to traverse the disc annulus between the superior vertebral body and the inferior vertebral body, such that
the first surface and the second surface are biased to diverge from the wall.

31. The support of claim 31, further comprising:
a fusion plate traversing an adjacent intervertebral disc space; and
a flexible member coupling the fusion plate and the wall of the support.

32. The support of claim 31, wherein the wall comprises at least one window such that at least one suture can anchor the support.

33. The support of claim 31, wherein the first surface and the second surface have curved edges.

34. The support of claim 31, wherein the first surface and the second surface have straight edges.

35. The support of claim 31, wherein at least one of the first surface, the wall, and the second surface includes a shaped memory alloy to bias the first surface and the second surface.

36. The support of claim 31, wherein the wall comprises an insert such that scarring material facilitates tissue growth.

37. The support of claim 37, wherein the scarring facilitates ligament reconstruction.

38. The support of claim 37, wherein the scarring anchors the wall to the disc annulus.
